(19) United States
(12) Patent Application Publication
(10) Pub. No.: US 2007/0191717 A1
(43) Pub. Date: Aug. 16, 2007

(54) CATHETER BASED IMPLANTED WIRELESS PRESSURE SENSOR

(75) Inventors: Arye Rosen, Cherry Hill, NJ (US); Paul Walinsky, Wyndmoor, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: DREXEL UNIVERSITY, Philadelphia, PA (US)

(21) Appl. No.: 11/674,426

(22) Filed: Feb. 13, 2007

Related U.S. Application Data
(60) Provisional application No. 60/772,774, filed on Feb. 13, 2006.

(51) Int. Cl. A61B 5/02

(52) U.S. Cl. 600/485; 607/122

(57) ABSTRACT

A catheter based implantable wireless pressure sensor and associated electronic circuitry for transmission of hemodynamic status of a subject.

### CROSS-REFERENCE TO RELATED APPLICATIONS

This utility application claims the benefit under 35 U.S.C. §119(e) of Provisional Application Ser. No. 60/772,774 filed on Feb. 13, 2006 entitled CATHETER BASED IMPLANTED WIRELESS PRESSURE SENSOR and whose entire disclosure is incorporated by reference herein.

### FIELD OF INVENTION

The present invention relates to a catheter based implantable wireless pressure sensor for determination of the hemodynamic status of a subject. This device more effectively allows long term monitoring of a subject's hemodynamic status in an ambulatory setting. This device is particularly useful in patients with congestive heart failure.

### BACKGROUND OF THE INVENTION

The National Institutes of Health have identified the diseases of congestive heart failure (CHF) and pulmonary hypertension as a treatment priorities in the United States (NIH website nhlbi with the extension nih/gov/health/public/heart/other/CHF.htm on the world wide web). CHF is characterized as a failure of the heart to pump blood efficiently. CHF affects half a million people in the United States alone, with an estimated cost of $40 million per year. The fatality rate from CHF is very high, with one in five patients dying within one year from the time of diagnosis, and more than half of CHF patients dying within 5 years (NIH website nhlbi with the extension nih/gov/health/public/heart/other/CHF.htm on the world wide web; 2002 Heart & Stroke Statistical Update. American Heart Association). Statistics for the young population are also alarming. A person of age 40 or above has a one in five chance of developing congestive heart failure (NIH website nhlbi with the extension nih/gov/health/public/heart/other/CHF.htm on the world wide web; 2002 Heart & Stroke Statistical Update. American Heart Association; Zeng et al. Hun XI Yi Ke Da Xue Bao 2000 31(2)246-247,259; Huonker et al. Cardiovasc. Drug. Ther. 1999 13:3233-241).

Another important clinical need for intracardiac pressure monitoring is in the patient with pulmonary hypertension. Research in the areas of congestive heart failure and pulmonary hypertension have resulted in new drugs and devices that are effective at improving symptomology and in increasing survival. However, proper pharmacological management requires knowledge of a patient's hemodynamic status.

Traditionally, assessing hemodynamic status of a patient is performed by examination of the patient and observation of the jugular venous pressure, the presence of abnormal heart sounds and the presence of edema in the lungs or in the extremities.

A more accurate means of determining a patient's hemodynamic status is direct measurement of pressure in the patient's heart. However, this is an invasive procedure that requires the placing of a catheter in the heart. Further, it is limited in terms of the duration that the catheter can be left in place.

All references cited herein are incorporated herein by reference in their entireties.

### BRIEF SUMMARY OF THE INVENTION

The present invention provides a pressure-sensing device for permanent catheter based implantation into the heart which is capable of assessing the hemodynamic status of a subject.

### BRIEF DESCRIPTION OF SEVERAL VIEWS OF THE DRAWINGS

The invention will be described in conjunction with the following drawings in which like reference numerals designate like elements and wherein:

FIG. 1 represents sensor and an oscillator circuit in an exemplary catheter based pressure sensing system.

FIG. 2 is a block diagram of an exemplary catheter based pressure sensing system.

### DETAILED DESCRIPTION OF THE INVENTION

The present invention provides a catheter based pressure measurement system for long-term implantation in a subject. In simplest form, this device comprises an accurate pressure sensor, a secure means of positioning the sensor in the heart, a means of transmission of data monitored by the sensor to a sensing and recording device outside of the heart and an energy source. This system is both biocompatible and stable in the body for a long period of time.

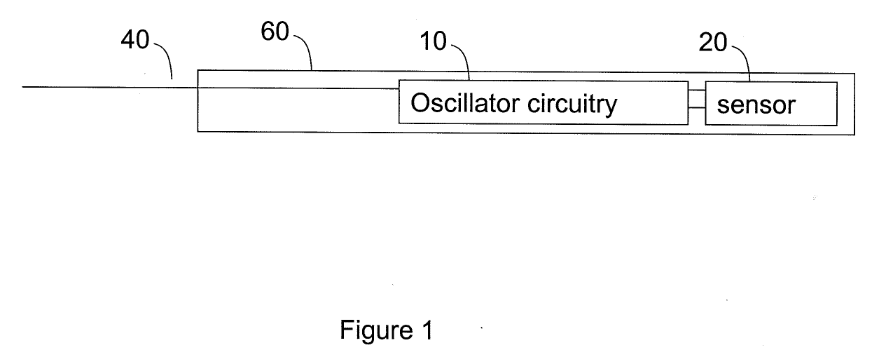

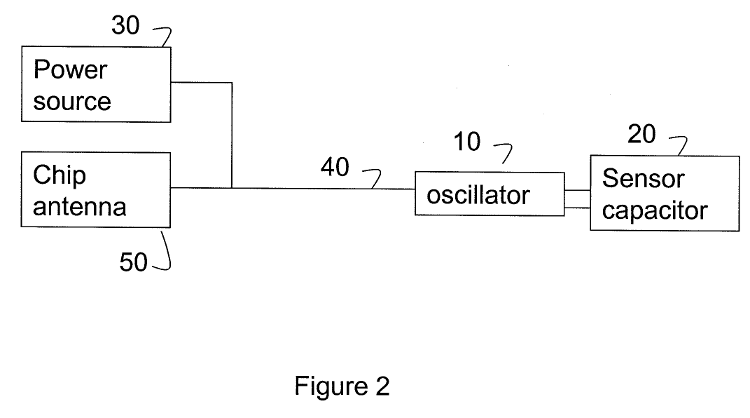

With reference to FIGS. 1 and 2, the accurate pressure sensor used in the present invention comprises a sensor core 10 and sensing component 20 enclosed in a catheter 60 and connected to a power source 30 thorough a coaxial cable 40, which also connects to a chip antenna, 50.

The sensor core 20 is preferably an oscillator operating at the Industrial-Scientific Medical (ISM) band of 2.4000-2.4835 GHz. It is also preferable to use a differential oscillator as opposed to a traditional Colpitts oscillator since the core transistors require four times lower bias current for oscillation. The microwave signal generated by the oscillator, whose oscillation frequency is directly related to the pressure, is radiated by an antenna embedded in the chest cavity, and is monitored by an external monitoring unit.

An oscillator based implantable unit operating at microwave frequency is preferred since oscillator frequency of a well-designed oscillator is very sensitive to the change of its tank capacitor. Further, the low frequency RF sensors operating at MHz range require that the receiving coil be properly aligned with the transmitting coil and be placed next to the patient's body. In contrast, a microwave signal transmitted by a small antenna inside the exterior of the chest cavity can be detected from a distance without compromising the patient's comfort. In addition, a microwave frequency of 2.4 GHz is high enough to be efficiently radiated by a small size antenna but is sufficiently low not to face significant absorption by the implant package and skin. Further, the huge market for wireless local networks and personal communication services, which operate at the same frequency range, has resulted in dramatic reductions in costs for this well-developed technology.

The sensing component is preferably a capacitor, whose capacitance variation with blood pressure changes the oscillation frequency of the oscillator. In one embodiment, the sensing component is a microelectromechanical system (MEMS) capacitor, whose capacitance changes with the deflection of its boron-doped membrane by the blood pressure. An exemplary sensor with a radius of 250 um has a nominal capacitance of 1.4 pF at 0 torr. The capacitive pressure sensor comprises a deflectable membrane that seals the cavity with reference pressure. The sensor measures the difference between pressure inside the cavity and outside pressure by the deflection of the membrane that serves as a plate of the capacitor. The deflection of the membrane causes capacitance to change accordingly. The change in capacitance will in turn change the resonant frequency of the LC tank of which the sensor capacitor is a part.

A capacitive method of pressure sensing is preferred over piezoresistive, piezoelectric or other approaches since capacitive pressure sensors are highly sensitive, rugged and extremely reliable. In addition, these devices show excellent resistance to both shock and vibration, and also have low power consumption. Further, capacitive pressure sensors can be fabricated from biocompatible materials such as silicon dioxide or aluminum oxide substrates and/or encapsulated in a thin layer of methylmethacrylate, which is easily cast and is already FDA approved for permanent implantation in neurosurgical procedures.

To conserve power required from the energy source, it is preferred that operation of the oscillator not be continuous in time but rather that the oscillator comprise a bias control which can be switched on and off periodically. For example, the bias control can be set to switch on and off periodically, with a period of T−1 ms and a pulse width of T0=0.1 ms. In this embodiment, the oscillation starts around 10 ns. Thus, a turn on duration of 0.1 ms corresponds to about 220 cycles, which is more than enough for detection purposes. Further, MOS transistors are preferably used, operating in a in weak inversion region (Stotts, L. J. IEEE Circuits and Devices Magazine 1989 5(1):12-18) for saving battery power. A period of 1 ms is generated by a three stage ring oscillator (Razavi, B. Design of Integrated Circuits for Optical Communications, New York, McGraw Hill 2002). The duty cycle of T0/T=0.0001 corresponds to an average current of 1.1 mA for the microwave oscillator, which is much lower than the rest of the CMOS circuitry (including pacemaker circuitry typically operating at around 20 mA (Stotts, L. J. IEEE Circuits and Devices Magazine 1989 5(1):12-18)). Short 0.1 ms pulses are generated through an RC circuit and a pair of invertors. During To a driving switch is on and turns on the microwave oscillator bias.

The pressure sensor of the device of the present invention is preferably sized to be less than 1 cubic centimeter so that it is small enough to be implanted in a catheter system (i.e. a Catheter Based Pressure Sensor) and permanently positioned in the heart. Because of the small thickness of the sensing mechanism, the device of the present invention can be integrated with a catheter used for pacemakers. Recent development of ICD's and biventricular pacemakers has made use of the pacemaking devices common in patients with abnormal heart function and heart failure. Incorporation of a pressure sensing device of the present invention into such a pacemaker provides a more effective means for monitoring patients with heart failure or pulmonary hypertension. The device of this invention can also be implanted independent of a pacemaker.

A means of transmission of data monitored by the sensor to a sensing and recording device outside of the heart and an energy source is provided via a miniature size coaxial cable 40 (1 mm in diameter) running through the catheter to a chip antenna 50 placed at the exterior of the chest cavity and, in patients with a pacemaker, adjacent to the pacemaker. The chip antenna is matched to the cable for efficient radiation of microwave signal.

This coaxial cable 40 is also connected to a power source 30, which supplies DC voltage to the pressure sensor. The power source can be independent or part of a pacemaker, with which the pressure sensor is associated. In another embodiment, the power source can be a miniature battery. In a still further embodiment, the power source can be a passive receiver for receiving microwave power from a source outside the patient's body.

The monitoring device preferably comprises a low noise amplifier, voltage control oscillators, mixers, filters and analog to digital converters. Preferably these components are in IC form. Frequency information is extracted using a baseband processing routine running on a computer and is transformed to pressure information.

The source of energy to power the device preferably comprises a battery such as that used in pacemakers. When used in a patient in conjunction with a pacemaker, a single source of power via the pacemaker's battery can be used.

A miniaturized membrane linked to a variable capacitor for use in the present invention was characterized. The capacitor was part of the oscillator's LC resonator, and its variation with the heart blood pressure changed the oscillation frequency of the oscillator.

While the invention has been described in detail and with reference to specific examples thereof, it will be apparent to one skilled in the art that various changes and modifications can be made therein without departing from the spirit and scope thereof.

What is claimed is:

1. An implantable apparatus for measuring hemodynamic status comprising:
(a) a pressure sensor having an electrical capacitance that varies with pressure;
(b) a pressure sensing circuitry connected to the pressure sensor for making a measurement of the capacitance of the pressure sensor;
(c) a wireless transmitter connected to the pressure sensing circuitry for transmitting the measurement of the pressure sensor.

2. The implantable apparatus of claim 1, further comprising:
a battery for powering the pressure sensing circuitry and the wireless transmitter, wherein the battery is connected to the pressure sensing circuitry and the wireless transmitter.

3. The implantable apparatus of claim 1, wherein:
the wireless transmitter and the active pressure sensing circuitry are powered by a microwave signal.

4. The implantable apparatus of claim 1, wherein:
the pressure sensor is enclosed in a catheter used for a cardiac pacemaker.
